(19) United States
(12) Patent Application Publication
(10) Pub. No.: US 2021/0307961 A1
(43) Pub. Date: Oct. 7, 2021

(54) COOLING NECK WRAP

(71) Applicant: Tracy Ian Seunath, Dayton, OH (US)

(72) Inventor: Tracy Ian Seunath, Dayton, OH (US)

(21) Appl. No.: 17/349,834

(22) Filed: Jun. 16, 2021

Related U.S. Application Data
(60) Provisional application No. 63/040,494, filed on Jun. 17, 2020.

(51) Int. Cl. A61F 7/10

(52) U.S. Cl. CPC A61F 7/103; A61F 2007/0011

(57) ABSTRACT

A neck wrap for cooling down a neck area of a person. The neck wrap can be worn around a neck and have two pouches. Each of the two pouches can receive a liquid container that can be filled with cold liquid and optionally frozen. The two pouches having a contact area coming in contact with skin made of heat conductive material that can permit removing heat from the neck area by the cold liquid in the liquid containers when the neck wrap is worn around the neck.

### CROSS-REFERENCE TO RELATED APPLICATIONS

This application claims priority to the U.S. provisional patent application Ser. No. 63/040,494, filed on Jun. 17, 2020, which is incorporated herein by reference in its entirety.

### FIELD OF INVENTION

The present invention relates to a neck wrap, and more particularly the present invention relates to a neck wrap for providing cooling around the neck area of a body.

### BACKGROUND

In warm and hot weather, people can easily get overheated while spending time outdoors. Excessive heat can cause discomfort and may become unbearable in hot weather. The body loses heat by sweating, however, in excessive heat, the natural thermoregulation mechanisms of the body may be insufficient. Without external ways to cool down the body, excessive heat can cause injury and can also affect the efficiency of a person.

Fans and air conditioning systems are used to cool down the body, however, such systems may not be available outdoors. The prior art lacks any solution to remove excessive heat around the neck area of a body. Thus, a desire is there for an effective device or an article of manufacture that can comfortably cool down the neck area in warm or hot weather.

### SUMMARY OF THE INVENTION

The following presents a simplified summary of one or more embodiments of the present invention in order to provide a basic understanding of such embodiments. This summary is not an extensive overview of all contemplated embodiments and is intended to neither identify key or critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to a neck wrap for cooling down the neck area of a person.

It is another object of the present invention that the neck wrap can be comfortably worn around the neck.

It is still another object of the present invention that the neck wrap is economical to manufacture.

It is yet another object of the present invention that the neck wrap is compact and portable.

It is a further object of the present invention that the neck wrap can work without power.

It is still another object of the present invention that the neck wrap can be easily carried around.

One general aspect includes a cooling neck wrap for removing heat from a neck and adjacent areas of a human or animal body. The cooling neck wrap includes a first pouch having a base and a wall that upstands from a periphery of the base, where the base and the wall define an inner volume of the first pouch, the wall of the first pouch has a contact area, the first pouch has an open-top configured to receive and retain a first liquid container. The cooling neck wrap also includes a second pouch having a base and a wall that upstands from a periphery of the base, where the base and the wall define an inner volume of the second pouch, the wall of the second pouch has a contact area, the second pouch has an open-top configured to receive and retain a second liquid container. The cooling neck wrap also includes a rear strap coupled at ends to the wall of the first pouch and the wall of the second pouch such as when the neck wrap is worn around a neck, the rear strap can extend along a rear side of the neck, the first pouch and the second pouch are positioned on a left side and a right side of the neck, and the contact areas of the first pouch and the second pouch faces the neck. The cooling neck wrap also includes a chin strap that extends between the base of the first pouch and the base of the second pouch, the chin strap has a fastener for wrapping and unwrapping the neck wrap around the neck.

Implementations may include one or more of the following features. The cooling neck wrap wherein the cooling neck wrap further may include the first liquid container and the second liquid container. The first liquid container and the second liquid container are squeeze water bottles. The rear strap has two opposite short edges coupled to a periphery of the first pouch and the second pouch. The rear strap having a length adjusting means for adjusting a length of the rear strap. The rear strap has an innermost layer of moisture-wicking material. The contact areas of the first pouch and the second pouch are made from a material that is thermally conducting, areas of the first pouch and the second pouch other than the contact areas are made from a thermally insulative material. The open tops of the first pouch and the second pouch are configured to tighten around the first liquid container and the second liquid container received within the first pouch and the second pouch. Each the first pouch and the second pouch further may include a sleeve running along a periphery of the first pouch and the second pouch, a drawstring extends through the sleeve, where the drawstring can be loosened to widen the open top of the first pouch and the second pouch, and the drawstring can be tightened to narrow the open top of the first pouch and the second pouch. Alternatively, each the first pouch and the second pouch further may include a zipper that extends from a periphery of the open-top along a length of the first pouch and the second pouch, where unzipping the zipper divides the wall widening the open top. A dimension of the contact area of the first pouch and the second pouch is proportional to a neck area such as maximum contact of the contact area can be made with a skin when the neck wrap is worn around a neck. The chin strap may include a hook and loop fastener, the hook and loop fastener has a first member and a second member, the chin strap further may include an elongated strap that extends from the base of the first pouch, the first member of the hook and loop fastener is coupled to an inner side of a tip of the elongated member, the second member is coupled to the base of the second pouch.

One general aspect includes a method for cooling a neck area of a human or animal body. The method also includes wrapping the neck wrap around a neck of a body and inserting the liquid containers filled with liquid and frozen into the two pouches.

These and other objects and advantages of the embodiments herein and the summary will become readily apparent from the following detailed description taken in conjunction with the accompanying drawings.

### BRIEF DESCRIPTION OF THE DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1 shows an exemplary embodiment of the neck wrap, according to the present invention.

### DETAILED DESCRIPTION

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely for the purpose of illustrating the general principles of the invention, since the scope of the invention will be best defined by the allowed claims of any resulting patent.

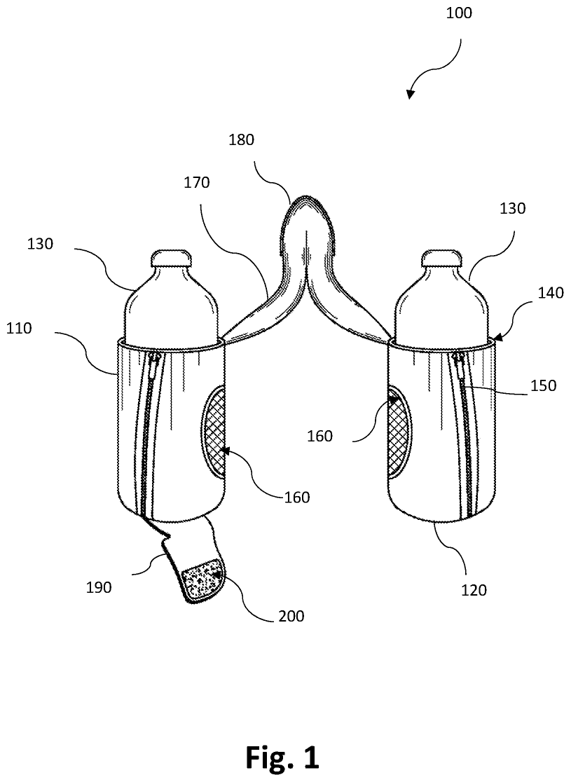

Disclosed is a neck wrap that can provide for cooling around the neck area of a person. The neck wrap can be worn around the neck, wherein the heat from the neck area can be removed by the cold liquid of the neck wrap. Referring to FIG. 1, the disclosed neck wrap 100 can include a pair of pouches i.e., a first pouch 110 and a second pouch 120. Each pouch can have a base and a wall that extends upwards from the base defining an inner volume of the pouch. The pouch can have a broad open-top that can receive a liquid container within the inner volume. FIG. 1 shows two water bottles 130 as the liquid containers within the first pouch and the second pouch. The water bottles can be interchangeably received into the pouch, such as the water bottle can be replaced by another bottle, or the water bottle can be removed and reinserted into the pouch. It is to be understood that liquid containers of different shapes and sizes are within the scope of the present invention, provided the liquid container can be received and retained within the pouch. The open-top 140 of the pouches can be loosened up to receive the container and tighten to retain the container within the pouch. For example, at least a periphery of the open-top of the pouch can be made of resilient material that can stretch to widen the opening and released over the liquid container to retain the liquid container. In another example, straps with fasteners can be provided along a periphery of the open-top, wherein the fasteners can be undone to widen the opening of the pouch and upon receiving the container, the fasteners can be tightened to retain the container. FIG. 1 shows a zipper 150 that extends from the periphery along the length of the pouch. Each pouch can be provided with one or two zippers on opposite sides. The zipper can be undone to widen the opening, such as the container can be received and upon receiving the container, the zipper can be zipped to tighten around the liquid container for retaining the liquid container. Still, alternatively, a drawstring can be provided along the periphery, wherein the drawstring can be tightened and loosen to widen or narrow the open-top. A sleeve can be provided along the periphery for the drawstring. Additionally, an elastic band can be provided that extends between opposite ends of the periphery of the pouch, such as the elastic band can pass over a liquid container in the pouch for preventing displacement of the liquid container from the pouch while working.

The liquid container can be a container that can be filled with liquid, such as water, wherein the liquid container having the filled liquid can be frozen. The liquid container can be rigid, semirigid, or flexible having a spout for filling the liquid. For example, the water bottles can be used, wherein water can be filled in the water bottle and the water bottle can then be frozen. The frozen water bottles can be inserted in the pouches of the neck wrap for cooling down the neck area. Preferably, a semi-rigid liquid container can be used that can retain its shape when empty or filled but can be squeezed under external pressure. For example, squeeze water bottles can be used.

The pouch can be made of flexible materials, such as neoprene. The area of the pouch coming in contact with the neck area can be made from a material having good thermal conductivity, such as the heat can be transferred from the neck area into the liquid contained in the bottle. Moreover, the materials in the contact area of the pouch can be skin-friendly and may also have moisture-wicking properties. The material of the contact area can prevent the sharp cold feeling on the neck, and the coolness can be distributed into the contact area for a soothing effect. FIG. 1 shows the contact area 160 made from a material different from the material of the pouch. The material of the pouch other than in the contact area 160 can be thermally insulative that can form a thermal barrier between the liquid container and the atmosphere. Thus, the liquid can be maintained at a low temperature for a longer time, and the majority of the heat can be absorbed through the contact area of the pouch from the neck area. The contact area can also be provided with suitable cushioning such as plush pads for comfort.

A rear strap 170 extends between the first pouch 110 and the second pouch 120. The opposite short edges of the rear strap 170 can be coupled to the periphery of the open-top 140 of the two pouches. The rear strap 170 can be sewn to the pouch. Alternatively, buckles, hooks, and the like fasteners can be used to couple the pouch to the rear strap. It is to be understood that the strap can be coupled to the periphery of the pouch or below the periphery without departing from the scope of the present invention. The exact position of attachment can be optimized such as maximum contact can be made between the contact area of the pouch and the skin. The rear strap 170 can have the elasticity to adjust to the variations in neck sizes of different people. Moreover, a larger size rear strap can be provided with size adjustment means, such as clip, buckle, clamp, and like to adjust the length of the rear strap according to variable sizes of the necks for a comfortable fit. FIG. 1 shows a clip 180 clamped to a fold of the rear strap to decrease its length. The rear strap can have an inner layer of moisture-wicking material that can absorb any sweat from the skin and provide a skin-friendly and comfortable feel to the wearer. Cushions can be added for additional comforts, such as a layer of plush cushion pads that can be provided behind the moisture-wicking layer.

A chin strap can also be provided for wrapping the neck wrap around the neck. The chin strap can extend between the base of the first pouch 110 and the base of the second pouch 120. The chin strap goes around the front of the neck for securing the two pouches in contact with the neck. The chin strap can have fasteners for wrapping and unwrapping the neck strap. The fasteners such as hook and loop fastener, snap buttons, buckles, and like fastening mechanism known to a skilled person for fastening a strap to a fixed point or fastening two straps are within the scope of the present invention. FIG. 1 shows an elongated strap 190 that extends from the base of the first pouch 110. The elongated strap 190 can have a first member 200 of the mating members of hook and loop fastener on the inner surface of the tip. The second member of the mating members can be provided on the base of the second pouch 120, such as the first member can be coupled to the second member for securing the elongated strap to the second pouch. Alternatively, a second elongated strap can also be provided that can extend from the base of the second pouch 120 and the second member of the mating member can be provided on an outer face of the second elongated strap. The second member can be elongated, to adjust the size of the neck wrap, wherein the first member of the mating members can be coupled at different positions on the second member.

It is to be understood that cooling the neck area does not mean restricting the area of the body that can be cooled. Perhaps the disclosed neck wrap can cool the face, chest, and back areas. Additional functionalities, such as reflector strips can also be provided. The reflector strips can be positioned on the outer surface of straps and the pouches. Also, the invention has been described as useful for cooling the neck area of a human body, however, the disclosed neck wrap can also be used for animals, such as a dog for providing cooling around the neck area in hot weather.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A cooling neck wrap for removing heat from a neck and adjacent areas of a human or animal body, the cooling neck wrap comprises:
a first pouch has a base and a wall that upstands from a periphery of the base, wherein the base and the wall define an inner volume of the first pouch, the wall of the first pouch has a contact area, the first pouch has an open-top configured to receive and retain a first liquid container;
a second pouch has a base and a wall that upstands from a periphery of the base, wherein the base and the wall define an inner volume of the second pouch, the wall of the second pouch has a contact area, the second pouch has an open-top configured to receive and retain a second liquid container;
a rear strap coupled at ends to the wall of the first pouch and the wall of the second pouch such as when the neck wrap is worn around a neck, the rear strap can extend along a rear side of the neck, the first pouch and the second pouch are positioned on a left side and a right side of the neck and the contact areas of the first pouch and the second pouch faces the neck; and
a chin strap that extends between the base of the first pouch and the base of the second pouch, the chin strap has a fastener for wrapping and unwrapping the neck wrap around the neck.

2. The cooling neck wrap according to claim 1, wherein the cooling neck wrap further comprises the first liquid container and the second liquid container.

3. The cooling neck wrap according to claim 2, wherein the first liquid container and the second liquid container are squeeze water bottles.

4. The cooling neck wrap according to claim 1, wherein the rear strap has two opposite short edges coupled to a periphery of the first pouch and the second pouch.

5. The cooling neck wrap according to claim 1, wherein the rear strap has a length adjusting means for adjusting a length of the rear strap.

6. The cooling neck wrap according to claim 1, wherein the rear strap has an innermost layer of moisture-wicking material.

7. The cooling neck wrap according to claim 1, wherein the contact areas of the first pouch and the second pouch are made from a material that is thermally conducting, areas of the first pouch and the second pouch other than the contact areas are made from a thermally insulative material.

8. The cooling neck wrap according to claim 1, wherein the open-tops of the first pouch and the second pouch are configured to tighten around the first liquid container and the second liquid container received within the first pouch and the second pouch.

9. The cooling neck wrap according to claim 8, wherein each the first pouch and the second pouch further comprises a sleeve running along a periphery of the first pouch and the second pouch, a drawstring extends through the sleeve, wherein the drawstring can be loosened to widen the open-top of the first pouch and the second pouch, and the drawstring can be tightened to narrow the open-top of the first pouch and the second pouch.

10. The cooling neck wrap according to claim 8, wherein each the first pouch and the second pouch further comprises a zipper that extends from a periphery of the open-top along a length of the first pouch and the second pouch, wherein unzipping the zipper divides the wall widening the open-top.

11. The cooling neck wrap according to claim 1, wherein a dimension of the contact area of the first pouch and the second pouch is proportional to a neck area such as maximum contact of the contact area can be made with a skin when the neck wrap is worn around a neck.

12. The cooling neck wrap according to claim 1, wherein the chin strap comprises a hook and loop fastener, the hook and loop fastener has a first member and a second member, the chin strap further comprises an elongated strap that extends from the base of the first pouch, the first member of the hook and loop fastener is coupled to an inner side of a tip of the elongated strap, the second member is coupled to the base of the second pouch.

13. A method for cooling a neck area of a human or animal body, the method comprising the steps of:
wrapping a neck wrap around a neck of a body, the neck wrap comprising:
a first pouch has a base and a wall that upstands from a periphery of the base, wherein the base and the wall define an inner volume of the first pouch, the wall of the first pouch has a contact area, the first pouch has an open-top configured to receive and retain a first liquid container,
a second pouch has a base and a wall that upstands from a periphery of the base, wherein the base and the wall define an inner volume of the second pouch, the wall of the second pouch has a contact area, the second pouch has an open-top configured to receive and retain a second liquid container,
a rear strap coupled at ends to the wall of the first pouch and the wall of the second pouch such as when the neck wrap is worn around a neck, the rear strap can extend along a rear side of the neck, the first pouch and the second pouch are positioned on a left side and a right side of the neck and the contact areas of the first pouch and the second pouch faces the neck; and
a chin strap that extends between the base of the first pouch and the base of the second pouch, the chin strap has a fastener for wrapping and unwrapping the neck wrap around the neck.

14. The method according to claim 13, wherein the first liquid container and the second liquid container are squeeze water bottles and the liquid is water.

15. The method according to claim 13, wherein the rear strap has two opposite short edges coupled to a periphery of the first pouch and the second pouch.

16. The method according to claim 13, wherein the rear strap has an innermost layer of moisture-wicking material.

17. The method according to claim 13, wherein the contact areas of the first pouch and the second pouch are made from a material that is thermally conducting, areas of the first pouch and the second pouch other than the contact areas are made from a thermally insulative material.

18. The method according to claim 13, wherein the open-tops of the first pouch and the second pouch are configured to tighten around the first liquid container and the second liquid container received within the first pouch and the second pouch.

19. The method according to claim 13, wherein a dimension of the contact area of the first pouch and the second pouch is proportional to a neck area such as maximum contact of the contact area can be made with a skin when the neck wrap is worn around a neck.

20. The method according to claim 13, wherein the chin strap comprises a hook and loop fastener, the hook and loop fastener has a first member and a second member, the chin strap further comprises an elongated strap that extends from the base of the first pouch, the first member of the hook and loop fastener is coupled to an inner side of a tip of the elongated strap, the second member is coupled to the base of the second pouch.
